(19) United States
(12) Patent Application Publication
(10) Pub. No.: US 2022/0080062 A1
(43) Pub. Date: Mar. 17, 2022

(54) Sanitizing sunshade

(71) Applicant: Kevin Shafer, Carlsbad, NM (US)

(72) Inventor: Kevin Shafer, Carlsbad, NM (US)

(21) Appl. No.: 17/018,249

(22) Filed: Sep. 11, 2020

(51) Int. Cl. A61L 2/10; B60J 1/20

(52) U.S. Cl. CPC A61L 2/10; A61L 2202/11; B60J 1/2011

(57) ABSTRACT

The present invention is directed to a sunshade for vehicles which has both a disinfecting and illuminating function. The sunshade includes a reflective outer surface and is foldable for stowing purposes. The inner surface includes at least a pair of UV lamps positioned thereon. The lamps, which may use UV-C radiation are preferably powered by both an attached solar cell array, and the vehicles' electrical system. When the vehicle is parked, the sunshade may be deployed within the windshield and electrically connected to the vehicle's battery. The UV lamps give off radiation which disinfects the vehicle while unoccupied. The lamps also give off colored radiation which has a pleasing aesthetic, especially at night.

### BACKGROUND OF THE INVENTION

### 1. Field of the Invention

This invention relates invention relates to automotive accessories. More particularly, it relates to a sunshade for a vehicle that can be used to disinfect and illuminate the interior of a vehicle.

### 2. Description of the Prior Art

Sunshades for placing within the windshields and other windows of automobiles and protecting the interiors of those automobiles against solar heating and sun damage have become popular, particularly in geographic regions such as the American southwest where the searing desert summer sun can quickly raise the interior temperature of a parked automobile to well over 120° F. This type of heating can prematurely age, fade, and otherwise damage the interior of an automobile. Folding sunshades made of cardboard, sometimes covered with a reflecting outer surface such as Mylar and plastic bubble wrap, are popular, but these are single function items. A number of more elaborate sunshades have also been proposed and/or sold.

U.S. Pat. No. 5,649,584 discloses a sunshade for vehicle windshields which includes a plurality of hinge sections which are foldable relative to one another to form a compact unit for storage and which are horizontally extendible when deployed across a substantial width of the vehicle windshield. Each section includes a fixed base panel member and a vertical sliding member which is extendible outwardly relative to the fixed member so that the sunshade is effectively adjustable for the height of a particular vehicle windshield.

U.S. Pat. No. 7,252,321 discloses a car sunshade includes: a pair of curtain members each hung to a hanger and operatively driven by a driving device to be upwardly unwound for shielding the windshield of the car from being exposed to sun light along a pair of guiding devices respectively mounted on (or juxtapositioned to) two pillars adjacent to the windshield; or the curtain members being downwardly wound to unveil the windshield of the car, without removing a factory-installed rear-view mirror and without obstructing a driver's head.

While these and other prior art sunshades are generally effective for their intended purposes, they are singular in their utility.

The present invention is directed to a sunshade for vehicles which has both a disinfecting and illuminating function. The sunshade includes a reflective outer surface and is foldable for stowing purposes. The inner surface includes at least a pair of UV lamps positioned thereon.

The lamps, which may use UV-C radiation are preferably powered by both an attached solar cell array, and the vehicles' electrical system. When the vehicle is parked, the sunshade may be deployed within the windshield and electrically connected to the vehicle's battery. The UV lamps give off radiation which disinfects the vehicle while unoccupied. The lamps also give off colored radiation which has a pleasing aspheric, especially at night.

### SUMMARY OF THE INVENTION

The present invention is directed to a sunshade for vehicles which has both a disinfecting and illuminating function. The sunshade includes a reflective outer surface and is foldable for stowing purposes. The inner surface includes at least a pair of UV lamps positioned thereon. The lamps, which may use UV-C radiation are preferably powered by either an attached solar cell array, or the vehicles' electrical system. When the vehicle is parked, the sunshade may be deployed within the windshield and electrically connected to the vehicle's battery. The UV lamps give off radiation which disinfects the vehicle while unoccupied. The lamps also give off colored radiation which has a pleasing aesthetic, especially at night.

It is a major object of the invention to provide an improved automotive sunshade.

It is another object of the invention to provide an improved automotive sunshade that can disinfect the vehicle interior.

It is another object of the invention to provide an improved automotive sunshade that uses UV lighting to disinfect a vehicle interior.

It is another object of the invention to provide an improved disinfecting automotive sunshade that can be powered by the electrical system of the vehicle.

It is another object of the invention to provide an improved disinfecting automotive sunshade that can be solar powered.

It is another object of the invention to provide an improved disinfecting automotive sunshade that can be self powered.

### BRIEF DESCRIPTION OF THE DRAWINGS

FIG. 1 shows a perspective view of a sunshade assembly formed in accordance with the invention, viewed from the exterior of the vehicle.

FIG. 2 shows a perspective view of a sunshade assembly formed in accordance with the invention, viewed from the interior of the vehicle.

FIG. 3 shows a perspective view of a sunshade assembly formed in accordance with the invention illustrating the illumination provided by the UV lamps.

FIG. 4 shows a plan view, with electronics connected, of a disinfecting mat to be used with the sunshade assembly of the invention.

### DETAILED DESCRIPTION OF THE PREFERRED EMBODIMENTS

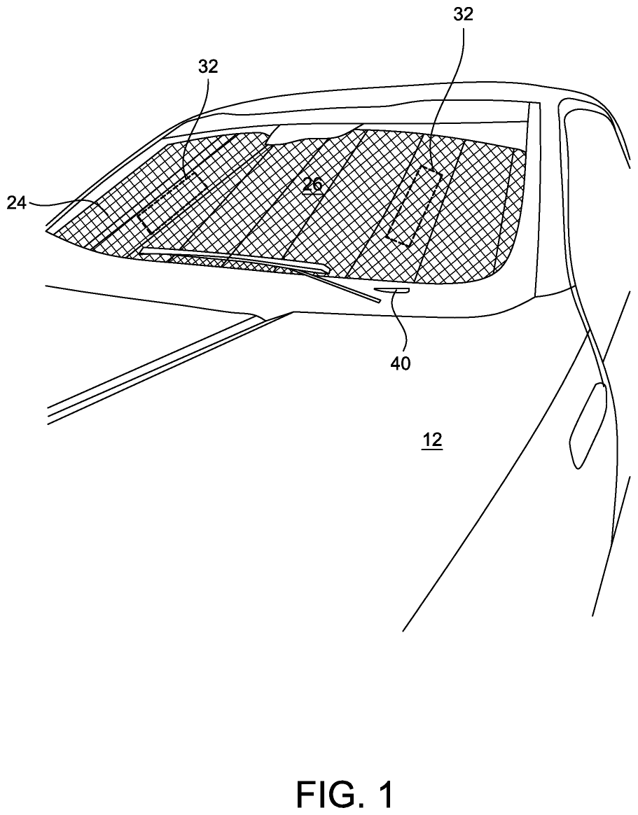

Referring now to the drawings, FIG. 1 illustrates the inventive disinfecting sunshade assembly, generally indicated by the numeral 10, positioned within a vehicle. The assembly 10 has the ability to disinfect, using UV radiation, a good part of the vehicle 12 interior 14, including any airborne aerosolized particles. The UV radiation may be UV A, B, or C. The assembly 10 also provides additional interior lighting for the vehicle, the lighting having a decorative aspect. In one aspect of the invention, the sunshade assembly may be used to provide interior lighting for the vehicle without the use of UV lights, the assembly used to position lighting merely for decorative effect.

Ultraviolet germicidal irradiation (UVGI) is a disinfection method that uses short-wavelength ultraviolet (UV-C) light to kill or inactivate microorganisms. One mechanism by which UV-C deactivates microorganisms is by destroying nucleic acids and disrupting their DNA, leaving them unable to perform vital cellular functions. The administration of UV-C radiation is becoming widely adopted by many hospitals as a more effective and reliable means of surface disinfection, as compared to the use of chemical cleaning agents alone. The effectiveness of germicidal UV-C irradiation depends on factors such as the length of time a microorganism is exposed to UV-C, the intensity and wavelength of the UV-C radiation, the presence of particles that can protect the microorganisms from UV, and a microorganism's ability to withstand UV-C during its exposure. In air and surface disinfection applications, a UV-C radiation source will disinfect the air and surfaces of the vehicle 12 interior which receive the radiation. In light of the Covid-19 pandemic, there is now a great need to disinfect all areas and surfaces that are exposed to people (or objects), especially in an enclosed environment like a vehicle interior.

Accordingly, the present invention is directed to an apparatus for disinfecting a vehicle 12 interior, which apparatus functions to provide both a sunshade and a uniquely aesthetic auxiliary interior light arrangement. The apparatus preferably uses UV-C radiation, but may also use UV-B or UV-A radiation.

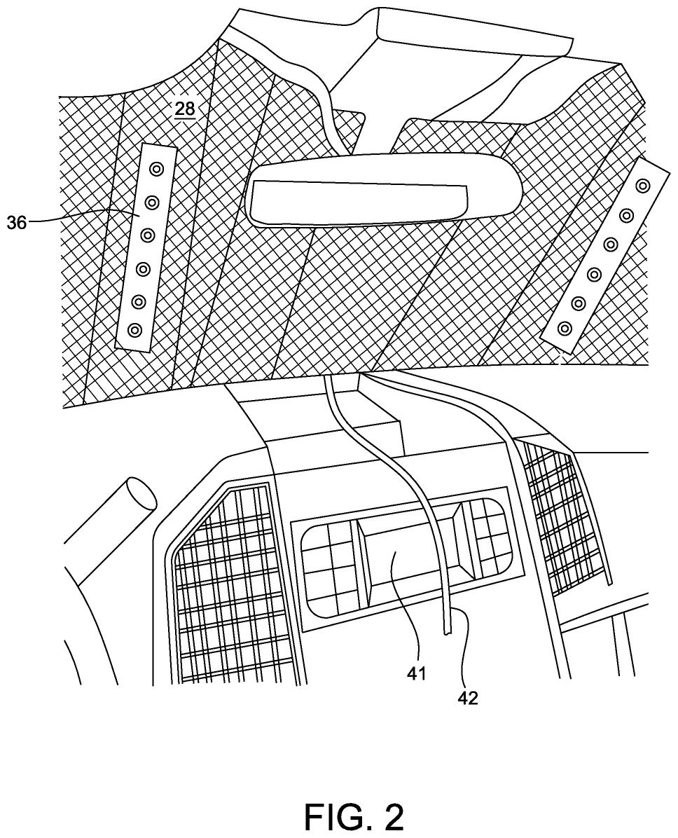

As can be seen more clearly in FIG. 2, the main body of the apparatus 10 is a more or less “standard” sunshade 20 which is foldable or collapsible for stowage. The collapsible sunshade 20 for a vehicle windshield (or window) is set forth, the sunshade 20 having a lateral and longitudinal dimension when in the extended state to block the rays of the sun from passing through the vehicle 12 windshield. The sunshade includes a plurality of lateral folds 22 defining panels 24 foldable in a superimposed, accordion-like fashion. The sunshade 20 has two sides, an outer facing side 26, and inner facing side 28. The sunshade 20 is made of cardboard or equivalent lightweight material. The outer facing side 26 is preferably highly reflective and may be covered with reflecting materials such as Mylar and plastic bubble wrap. The cardboard material used for the sunshade 20 may be layered so that internal (hidden) wiring is not visible.

Positioned on the outer side 26 are one or more solar panels 32, preferably of the lightweight “flexible” type so as not to add too much weight or bulk to the sunshade 20. The solar panels 32 are electrically connected to at least a pair of spaced UV light strips 36 by wires 34. Preferably, there is enough solar panel 32 square footage to provide enough wattage for the strips 36 to provide some disinfection, assuming a sufficient amount of ambient sunlight is available when deployed. Space is provided on the outer side 26 for warning indicia regarding the dangers of being irradiated by the UV radiation from strips 32. The space may also be used for advertising or other indicia.

The light strips 36 are regularly spaced regardless of number, so that the radiation emitted therefrom is distributed evenly throughout the interior of the vehicle. Optionally, 3 or more UV light strips 36 may be used, with one or more centrally positioned strips 36 illuminating the rear of the interior of the vehicle 12. As shown, both light strips 36 are positioned on the third panel 24 from their respective edges, but any number and positioning of the strips 36 that allows for an even distribution of UV radiation is contemplated by the invention. A warning label may be positioned on the sunshade 10 regarding the dangers of being irradiated with UV light.

The strips 36, which are preferably UV-C, are mounted onto the sunshade by overlying clear tape 37 through which the UV radiation is emitted. The tape is preferably UV resistant. The strips 36 may be positioned upon appropriately sized strips of highly reflective material 39 such as a reflective foil which is adhesively secured to the sunshade 20.

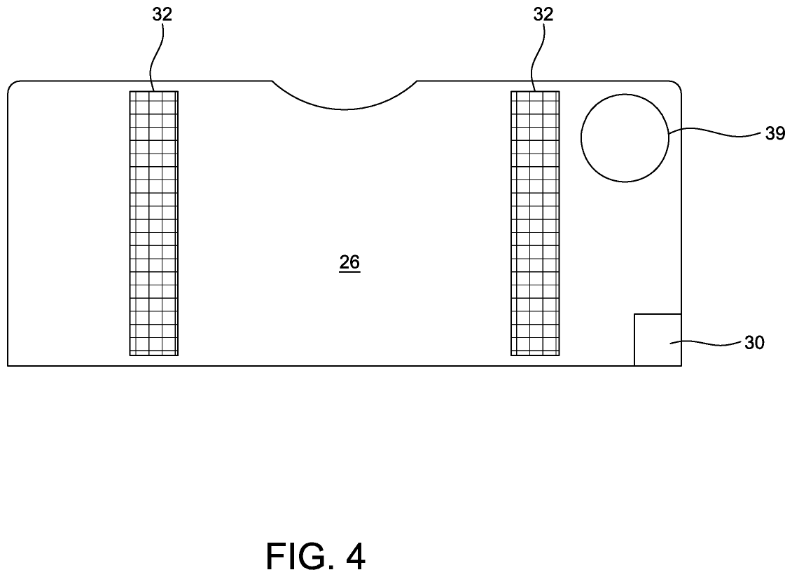

When in full power mode, the strips 36 must be connected to the vehicle battery 31 or electrical system as shown most clearly in FIG. 4. To that end, the strips 36 are connected to the vehicle auxiliary power outlet 41 via wires 42. In order to prevent excessive battery 31 drain, and to facilitate other processes, circuitry 30 is provided to automatically shut the strips 36 down after a predetermined amount of time, typically about 30 minutes. The circuitry or processor 30 is in the form of a microprocessor capable of receiving inputs from sensors and switching power to the strips 36. The processor 30 may be a self contained module appended to wire 42. Alternatively, processor 30 may be contained within the main body of the sunshade 20. The solar panel 32 is electrically connected to processor 30, the processor determining if there is sufficient power available from the solar panel to power the strips 36. If the power level from the solar panel 32 drops below that required for activation of the strips 32, processor 30 can switch to battery 31 power. Processor 30 can also condition and regulate the power output from the solar panel 32. Processor 30 may also include a sensor 39 for detecting a signal from a transmitting fob 34. Alternatively, the sensor 39 may be embedded in the main body of the sunshade, and electrically connected to processor 30. The fob 34 may be like that used for a keyless entry system, and transmits a low level activation signal which triggers a switch in the circuitry 30 which activates the strips 36. In this manner, the user is never irradiated while the strips 36 are active. Alternatively, a smartphone application can be downloaded, the application allowing the user to transmit a code to processor 30 for activating the lamps 36 using a smartphone. Methods for utilizing a smartphone application to allow a smartphone to communicate with processor 30 would be apparent to one of skill in the art. It should be appreciated that as processor 30 is connected by wire 42 to the apparatus 10 it can be extended some distance therefrom to receive a signal from the fob 34 or smartphone. Thus, if the sunshade 20 is disposed in a vehicle, the processor 30 and sensor 39 can be positioned near a window.

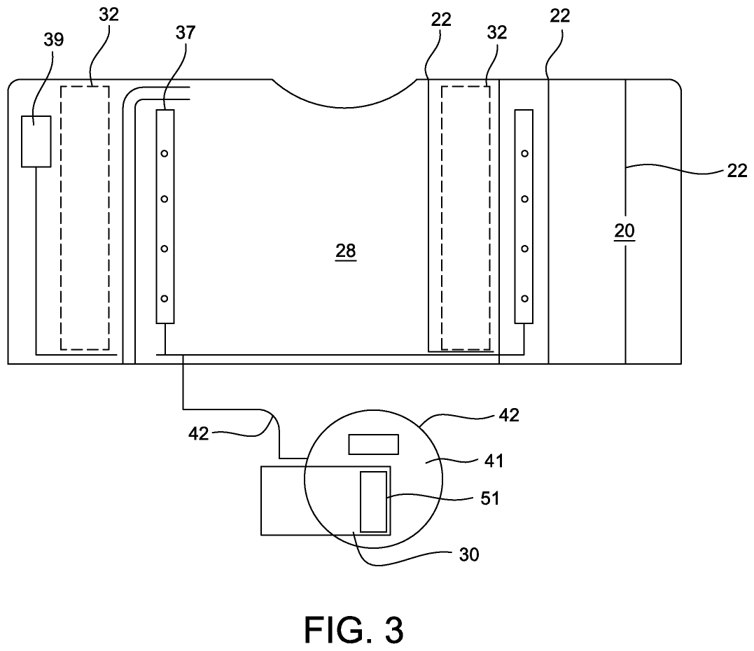

As UV lamps give off very bright and unusually colored light, the strips 36 may also be used to present an aesthetically pleasing interior lighting option. As can be seen especially in FIG. 3, the strips 36 may be seen from a distance and produce a striking visual. In an alternative embodiment, the strips 36 may be replaced by regular (non-UV) lighting, the sunshade serving to position the lighting for optimal visual effect. Solar panels 32 can be used to send an ambient light signal to processor 30, so that the strips 36 are illuminating at nighttime when the sunshade is connected to battery 31 and deployed as shown in FIG. 1

An additional disinfecting UV source may be combined with the sunshade 20. The source is in the form of a mat 50 which can be self powered or preferably electrically connected to battery 31 via processor 30. The mat 50 may be connected to processor 30, which may include an on/off switch 51 in addition to the activation sensor 39 as described above, can be used to selectively power the mat 50 whenever power is applied to sunshade 20. If one or more mats 50 are used they may all be connected to battery 31 through processor 30, this allowing processor 30 to control power to the sunshade 20 and all mats 50 simultaneously. Each mat 50 includes one or more light strips 36 positioned about the edges of the mat 50, so as to give the best possible light distribution, the strips held in place using clear tape as explained above. The mat 50 may be positioned as desired upon the floor, seat, or even the interior of the roof of the vehicle, with more than one mat 50 used if necessary or desired to disinfect the entire interior of the vehicle. The mat 50 may be used as a floormat exclusively, all floormats in a given vehicle may be replaced by the inventive floormat. The mats 50 may use non UV lighting for an aesthetic effect.

In use, the vehicle operator can unfold and position the sunshade 20 as shown in FIG. 1 so that the entire windshield 40 is covered and appropriate shading is effected. The strips 36 may be activated by way of on/off switch 51 on circuit 30 or by using the remote activation method as discussed above. The optional mat 50 is activated when the sunshade 20 is activated.

It is to be understood that the present invention is not limited to the sole embodiment described above, but encompasses any and all embodiments within the scope of the following claims:

I claim:

1. A disinfecting sunshade assembly for an automobile having a windshield comprising:
a flat planar main body having an inner and outer side, said main body characterized by a series of vertical folds, said folds allowing for the creation of a number of panels;
at least a pair of UV light strips to attached to said panels, each of said UV light strips entirely positioned on a single one of said panels;
said UV light strips electrically connected to a source of electrical power.

2. The sunshade assembly of claim 1 wherein said automobile has a battery, and said source of power is said battery.

3. The sunshade assembly of claim 1 wherein said outer side has at least one solar panel positioned thereon.

4. The sunshade assembly of claim 3 wherein said at least one solar panel is said source of power.

5. The sunshade assembly of claim 1 including a processor for controlling application of power from said source of power to said at least pair of UV light strips.

6. The sunshade assembly of claim 5 wherein said at least one solar panel is electrically connected to said processor.

7. The sunshade assembly of claim 6 wherein said processor determines power output from said at least one solar panel.

8. The sunshade assembly of claim 1 including a mat electrically connected to said source of power and having UV lighting attached thereto, said mat positionable anywhere within said automobile.

9. The sunshade assembly of claim 8 wherein said UV lighting is powered by said source of power or said at least one solar panel.

10. The sunshade assembly of claim 1 wherein said light strips are UVA, UVB, or UVC.

11. A sunshade assembly for an automobile having a windshield comprising:
a flat planar main body having an inner and outer side, said main body characterized by a series of vertical folds, said folds allowing for the creation of a number of panels;
at least a pair of light strips to attached to said panels, each of said light strips entirely positioned on the inner side of a single one of said panels;
said light strips electrically connected to a source of electrical power.
